(19) United States
(12) Patent Application Publication
(10) Pub. No.: US 2007/0191652 A1
(43) Pub. Date: Aug. 16, 2007

(54) Process for production of 1,1,1,2- tetrafluoroethane and/or pentafluorethane and applications of the same

(76) Inventors: Hiromoto Ohno, Kanagawa (JP); Toshio Ohi, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/591,119

(22) PCT Filed: Mar. 28, 2005
(86) PCT No.: PCT/JP05/06521

§ 371 Date: Aug. 30, 2006

Related U.S. Application Data
(60) Provisional application No. 60/559,428, filed on Apr. 6, 2004.

(30) Foreign Application Priority Data
Mar. 29, 2004 (JP) 2004-095012

(51) Int. Cl. C07C 19/08; C07C 17/08

(52) U.S. Cl. 570/165

(57) ABSTRACT

A process for producing high purity 1,1,1,2-tetrafluoroethane and/or pentafluoroethane by the step of purifying a crude product obtained by reacting trichloroethylene and/or tetrachloroethylene with hydrogen fluoride comprised of a main product including 1,1,1,2-tetrafluoroethane and/or pentafluoroethane, hydrogen fluoride as an azeotropic component with the main product, and impurity ingredients including at least an unsaturated compound, wherein said purifying step includes a step of bringing a mixture obtained by newly adding hydrogen fluoride into said crude product into contact with a fluorination catalyst in the vapor phase to reducing the content of the unsaturated compound contained in said crude product and a distillation step.

### CROSS-REFERENCE TO RELATED APPLICATION

This application is an application filed under 35 U.S.C. §111(a) claiming benefit pursuant to 35 U S.C. §119(e)(1) of the filing date of the Provisional Application 60/559,428 filed Apr. 6, 2004, pursuant to 35 U.S.C. §111(b).

### TECHNICAL FIELD

The present invention relates to a process for producing 1,1,1,2-tetrafluoroethane and/or pentafluoroethane and applications of the same.

### BACKGROUND ART

As methods of production of 1,1,1,2-tetrafluoroethane (HFC-134a or CF₃CH₂F) and pentafluoroethane (HFC-125 or CF₃CHF₂), conventionally the following methods are known.

As a method of production of 1,1,1,2-tetrafluoroethane, for example the method of production by reacting trichloroethylene and hydrogen fluoride in the presence of a fluorination catalyst is known Further, as a method of production of pentafluoroethane, the method of production by reacting tetrachloroethylene and hydrogen fluoride in the presence of a fluorination catalyst is known. When producing 1,1,1,2-tetrafluoroethane and pentafluoroethane by these methods, various impurities are produced as byproducts according to the reaction conditions used. These impurities include unsaturated compounds such as CF₂═CClF, CF₂═CHCl, CHF═CClF, CClF═CHCl, CHCl═CHF, CF₂═CHF, and CF₂═CClF and include chlorofluorocarbons such as CCl₂F₂, CH₂ClF, CH₂ClCClF₂, CF₃CHCl₂, and CF₃CClF₂ and hydrofluorocarbons such as CH₂F₂, CF₃CH₃, and CHF₂CHF₂.

Among these impurities, hydrofluorocarbons do not cause any problem so far as they are small in amount, but it is necessary to reduce the contents of the unsaturated compounds and the chlorofluorocarbons as much as possible. They can be removed to a certain extent by fractional distillation etc. However, it is extremely difficult to remove impurities having boiling points close to those of 1,1,1,2-tetrafluoroethane and pentafluoroethane to a low enough level that they substantially do not exist by fractional distillation. It is also difficult to remove impurities forming an azeotropic composition and an azeotropic-like composition in the same way as the former. For this reason, various processes have been proposed as the method for solving this problem.

For example, a method of purifying unsaturated compounds (mainly CF₂═CHCl) included as impurities in crude CF₃CH₂F from which hydrogen chloride has been removed to a certain extent by reacting it with hydrogen fluoride as an azeotropic component with CF₃CH₂F in the presence of a fluorination catalyst (Japanese Unexamined Patent Publication No. 6-184015) is known. However, this method leaves behind technical problems such as the production of unsaturated compounds by the dehalogenation reaction of the intermediate 2-chloro-1,1,1-trifluoroethane (CF₃CH₂Cl) contained in the target CF₃CH₂F and the shortened life of the fluorination catalyst.

### DISCLOSURE OF INVENTION

The problem to be solved by the present invention is to provide a novel process for production of 1,1,1,2-tetrafluoroethane and/or pentafluoroethane for solving the problems of the prior art described above and applications for the same.

In consideration with the above circumstances, the inventors engaged in intensive studies so as to develop a process for production of 1,1,1,2-tetrafluoroethane and/or pentafluoroethane which can be industrially worked and which is economical and as a result found out that the above problem could be solved by using a process for producing high purity 1,1,1,2-tetrafluoroethane and/or pentafluoroethane by a step of purifying a crude product obtained by reacting trichloroethylene and/or tetrachloroethylene with hydrogen fluoride comprised of a main product including 1,1,1,2-tetrafluoroethane and/or pentafluoroethane, hydrogen fluoride as an azeotropic component with the main product, and impurity ingredients including at least an unsaturated compound, wherein said purifying step includes a step of bringing a mixture obtained by newly adding hydrogen fluoride into said crude product into contact with a fluorination catalyst in the vapor phase to reducing the content of the unsaturated compound contained in said crude product and a distillation step, and thereby completed the present invention.

Namely, the present invention includes the means of for example the following (1) to (13).

(1) A process for producing high purity 1,1,1,2-tetrafluoroethane and/or pentafluoroethane by a step of purifying a crude product obtained by reacting trichloroethylene and/or tetrachloroethylene with hydrogen fluoride comprised of a main product including 1,1,1,2-tetrafluoroethane and/or pentafluoroethane, hydrogen fluoride as an azeotropic component with the main product, and impurity ingredients including at least an unsaturated compound, wherein said purifying step includes a step of bringing a mixture obtained by newly adding hydrogen fluoride into said crude product into contact with a fluorination catalyst in the vapor phase to reducing the content of the unsaturated compound contained in said crude product and a distillation step.

(2) A production process as set forth in (1), wherein the content of the hydrogen chloride contained as the impurity in said crude product is 2 mol % or less.

(3) A production process as set forth in (1) or (2), wherein the concentration of the 1,1,1,2-tetrafluoroethane and/or pentafluoroethane contained in said crude product is 70 mol % or more.

(4) A production process as set forth in any one of (1) to (3), wherein said unsaturated compound is at least one compound selected from a group consisting of 1,1-difluoro-2-chloroethylene, 1,2-difluoro-1-chloroethylene, 1-chloro-2-fluoroethylene, 1,1,2-trifluoroethylene, and 1-chloro-1,2,2-trifluoroethylene.

(5) A production process as set forth in any one of (1) to (4), wherein said fluorination catalyst includes at least one metal element selected from a group consisting of Cu, Mg, Zn, Pb, V, Bi, Cr, In, Mn, Fe, Co, Ni, and Al.

(6) A production process as set forth in any one of (1) to (5), wherein a contact temperature between said mixture and said fluorination catalyst is within a range of from 130 to 280° C.

(7) A production process as set forth in any one of (1) to (6), wherein a mixture obtained by newly adding hydrogen fluoride to a crude product comprised of a main product including 1,1,1,2-tetrafluoroethane, hydrogen fluoride as an azeotropic component with the main product, and impurity ingredients including at least an unsaturated compound is brought into contact with the fluorination catalyst in the vapor phase to reduce the content of the unsaturated compound contained in said crude product.

(8) A production process as set forth in (7), wherein the contact temperature between said mixture and said fluorination catalyst is within a range of from 130 to 200° C.

(9) A production process as set forth in any one of (1) to (8), further comprising separating the hydrogen fluoride in said distillation step and recirculating the separated hydrogen fluoride to a step for obtaining said crude product.

(10) A 1,1,1,2-tetrafluoroethane obtained by the production process as set forth in any one of (1) to (9), wherein a total content of chlorine-containing compounds in said 1,1,1,2-tetrafluoroethane is 2 volppm or less.

(11) A process for production of pentafluoroethane and/or hexafluoroethane characterized by reacting the 1,1,1,2-tetrafluoroethane as set forth in (10) and fluorine gas in the presence of a diluting gas.

(12) An etching gas comprising pentafluoroethane and/or hexafluoroethane obtained by the production process as set forth in (11).

(13) A cleaning gas comprising pentafluoroethane and/or hexafluoroethane obtained by the production process as set forth in (11).

According to the present invention, an industrially advantageous production process for obtaining 1,1,1,2-tetrafluoroethane and/or pentafluoroethane which can be advantageously utilized as a low temperature refrigerant, an etching gas, or a cleaning gas by reducing the content of unsaturated impurities contained in 1,1,1,2-tetrafluoroethane and/or pentafluoroethane and applications thereof can be provided.

### BEST MODE FOR CARRYING OUT THE INVENTION

Below, a detailed explanation will be given of the present invention.

As a process for production of CF₃CH₂F, for example a process for production by reacting trichloroethylene and hydrogen fluoride in the presence of a fluorination catalyst in two steps is known. Further, as a process for production of CF₃CHF₂, for example a process for production by reacting tetrachloroethylene and hydrogen fluoride in the presence of a fluorination catalyst in two steps is known. When producing CF₃CH₂F and CF₃CHF₂ by using these processes, even when purifying such as by the generally practiced distillation operation, impurities hard to separate from the target CF₃CH₂F and CF₃CHF₂ are contained. As these impurities, there can be mentioned for example the above unsaturated compounds, chlorofluorocarbons, hydrofluorocarbons, etc. It is necessary to remove these impurities as much as possible to obtain a high purity.

The process of production of 1,1,1,2-tetrafluoroethane and/or pentafluoroethane of the present invention is a process for producing high purity 1,1,1,2-tetrafluoroethane and/or pentafluoroethane by the step of purifying a crude product obtained by reacting trichloroethylene and/or tetrachloroethylene with hydrogen fluoride comprised of a main product including 1,1,1,2-tetrafluoroethane and/or pentafluoroethane, hydrogen fluoride as an azeotropic component with the main product, and impurity ingredients including at least an unsaturated compound, wherein said purifying step includes a step of bringing a mixture obtained by newly adding hydrogen fluoride into said crude product into contact with a fluorination catalyst in the vapor phase to reducing the content of the unsaturated compound contained in said crude product and a distillation step.

It is known that many compounds of hydrofluorocarbons form azeotropic mixtures with hydrogen fluoride. CF₃CH₂F and CF₃CHF₂ also form azeotropic mixtures together with hydrogen fluoride. For example, the molar ratio of the azeotropic mixture of CF₃CH₂F and hydrogen fluoride is HF/CF₃CH₂F=about 0.12. For example, the above Japanese Unexamined Patent Publication No. 6-184015 discloses a method of bringing a mixture including this azeotropic mixture and an unsaturated impurity such as 1,1-difluoro-2-chloroethylene (CF₂═CHCl) into contact with a chromium-based catalyst at a temperature of 200 to 380° C. to reduce the unsaturated compounds. However, this involved the problem that when the contact temperature became high, the dehalogenation reaction of the 2-chloro-1,1,1-trifluoroethane (CF₃CH₂Cl) contained in the mixture resulted in 1,1-difluoro-2-chloroethylene being produced as a byproduct, so coking of the catalyst surface proceeded along with that and the catalyst life became shorter. In the present invention, by newly incorporating hydrogen fluoride into a mixture including a main product including CF₃CH₂F and/or CF₃CHF₂, hydrogen fluoride as the azeotropic component with the main product, and one or more types of unsaturated compounds and bringing this mixture into contact with the fluorination catalyst in the vapor phase state, the advantages are obtained that the content of the unsaturated compounds is reduced without loss of the target product and further the catalyst life can be extended.

The CF₃CH₂F crude product obtained by reacting the trichloroethylene and the hydrogen fluoride, then performing the crude purifying step includes hydrogen fluoride as the azeotropic component, one or more types of unsaturated compounds, and CF₃CH₂Cl as an intermediate in production of the CF₃CH₂F. Usually, the concentration of the CF₃CH₂Cl is about 10 mol % or less, and the concentration of the target CF₃CH₂F is 70 mol % or more. The CF₃CH₂Cl of the intermediate forms an azeotropic mixture together with the hydrogen fluoride. The molar ratio of the azeotropic mixture is HF/CF₃CH₂Cl=about 1.0.

Further, the total content of the unsaturated compounds differs according to the catalyst and reaction conditions used, but generally is about 0.4 to 0.9 mol %. As the unsaturated compounds, there can be mentioned 1,1-difluoro-2-chloroethylene, 1,2-difluoro-1-chloroethylene, 1-chloro-2-fluoroethylene, 1,1,2-trifluoroethylene, and 1-chloro-1,2,2-trifluoroethylene. The molar ratio of the azeotropic mixture of the CF₃CH₂F and the hydrogen fluoride is HF/CF₃CH₂F=about 0.12, therefore the amount of the newly incorporated hydrogen fluoride preferably becomes more than this. This is preferably added so that the molar ratio with the CF₃CH₂F becomes HF/CF₃CH₂F=0.3 or more. When the amount of addition of the hydrogen fluoride is increased, the addition reaction of the hydrogen fluoride to the unsaturated compound easily progresses and the reaction temperature can be lowered. This results in the large advantages such as the suppression of the production of byproducts and the reduction of the loss of the target product and the prolonged life of the catalyst. Further, when the crude product is CF₃CHF₂, the molar ratio of the azeotropic mixture of the CF₃CHF₂ and the hydrogen fluoride is HF/CF₃CHF₂=about 0.1, so the hydrogen fluoride to be newly added is preferably added in an amount giving a molar ratio with the CF₃CHF₂ of HF/CF₃ CHF₂=0.2 or more. In the process of the present invention, the step of bringing the mixture obtained by newly adding hydrogen fluoride into the crude product into contact with a fluorination catalyst in the vapor phase may comprise mixing the CF₃CH₂F and the CF₃ CHF₂ alone with hydrogen fluoride to bring the mixture into contact with the fluorination catalyst or may form a mixture with the hydrogen fluoride in a state where two compounds are mixed and bring the mixture into contact with the fluorination catalyst. The method of bringing the mixture into contact with the fluorination catalyst in the state where two compounds are mixed, then distilling off and separating them is preferred. Further, as the method for feeding the hydrogen fluoride to be newly added, any of the complete feeding method and batch feeding method can be selected.

The fluorination catalyst used in the process of the present invention may be any having a catalytic action with respect to a fluorination reaction. As the catalyst, a fluorination catalyst comprised of a metal compound of Group IB, Group IIA, Group IIB, Group IVB, Group VA, Group VB, Group VIA, Group VIIA, and Group VIII of the Periodic Table including at least one type of element selected from the group consisting of Cu, Mg, Zn, Pb, V, Bi, Cr, In, Mn, Fe, Co, Ni, and Al, for example, a bulk catalyst comprised mainly of trivalent chromium oxide or a supported catalyst using alumina, aluminum fluoride, or active carbon as a carrier can be selected. As the method of preparation of the fluorination catalyst, the usual method can be applied. This can be produced by for example impregnating alumina with a cobalt chloride aqueous solution, drying it, then calcining it in a flow of air. The catalyst prepared in this way is preferably activated by using nitrogen and/or hydrogen fluoride before use for the reaction.

The temperature at which the crude product and the fluorination catalyst are brought into contact is preferably within a range of from 130 to 280° C., more preferably within a range of from 130 to 200° C. When the temperature is lower than 130° C., the reaction rate of the unsaturated compound tends to become slow, while at a temperature higher than 280° C., a tendency of increase of the ratio of the secondary reaction as described above is seen.

The content of the hydrogen chloride contained as an impurity in the crude product is preferably 2 mol % or less. When the content of the hydrogen chloride is larger than 2 mol %, the impurity tends to increase.

After bringing the crude product and the fluorination catalyst into contact, preferably the hydrogen fluoride of the azeotropic component and at least part of the newly added hydrogen fluoride are separated in the distillation step, and the separated hydrogen fluoride is recirculated to the step of obtaining the crude product. The CF₃CH₂F can be separated and purified by distillation, therefore high purity CF₃CH₂F including almost no unsaturated compound and chlorine-containing compounds can be obtained with a high yield. The total content of the chlorine-containing compounds can be reduced to 2 volppm or less.

The content of the impurities contained in the CF₃CH₂F can be measured by the TCD method or the FID method of gas chromatography (GC), the gas chromatography-mass spectrometer (GC-MS) method, etc.

Further, by reacting such high purity 1,1,1,2-tetrafluoroethane and fluorine gas in the presence of a diluting gas, pentafluoroethane and/or hexafluoroethane can be produced. According to the process for production of the present invention, the 1,1,1,2-tetrafluoroethane serving as the material for producing the pentafluoroethane and hexafluoroethane has an extremely small total content of chlorine-containing compounds contained as impurities, therefore high purity pentafluoroethane and hexafluoroethane can be produced. For example, the pentafluoroethane can be given a purity of 99.9998 vol % or more.

Next, an explanation will be given of applications of the high purity pentafluoroethane and hexafluoroethane obtained by using the above production process. The high purity pentafluoroethane may for example be mixed with an inert gas such as He, N₂, or Ar and a gas such as O₂ or NF₃ (hereinafter also referred to as a “pentafluoroethane product”) and used as an etching gas in an etching step in a semiconductor device production process. Further, the high purity hexafluoroethane can be used as a cleaning gas in a semiconductor device production process.

Below, the present invention will be further explained by examples and comparative examples, but the present invention is not limited to these examples.

### EXAMPLE OF PREPARATION OF CRUDE 1,1,1,2-TETRAFLUOROETHANE (MATERIAL EXAMPLE 1)

Using trichloroethylene (CCl₂═CHCl) as the starting material, a two-step reaction was performed of reacting this with hydrogen fluoride in the vapor phase in the presence of a chromium-based fluorination catalyst to mainly obtain an intermediate of CF₃CH₂Cl, introducing this into another reactor filled with the chromium-based fluorination catalyst, and further reacting this with hydrogen fluoride. The crude 1,1,1,2-tetrafluoroethane obtained after the crude purifying step was analyzed, whereupon it had the following composition:

| | | | |
|---|---|---|---|
| CF₃CH₂F | 81.2080 | CHCl═CHF | 0.0020 |
| CF₃CH₂Cl | 6.2400 | CF₃CH₃ | 0.5630 |
| CF₃CHF₂ | 0.5320 | CF₃CHClF | 0.5310 |
| CHF₂CHF₂ | 0.1600 | CF₃CClF₂ | 0.0540 |
| CF₂═CHCl | 0.6420 | HF (hydro- | 9.5060 |
| HCl (hydro- | 0.5620 | gen fluoride) | |
| gen chloride) | | | |
| | | | Unit: vol % |

### EXAMPLE OF PREPARATION OF CRUDE PENTAFLUOROETHANE (MATERIAL EXAMPLE 2)

Using tetrachloroethylene (CCl₂═CCl₂) as the starting material, a two-step reaction was performed of reacting this with hydrogen fluoride in a vapor phase in the presence of a chromium-based catalyst to mainly obtain CF₃CHCl₂ and CF₃CHClF as the intermediates and introducing this into another reactor filled with a chromium-based fluorination catalyst to react this with the hydrogen fluoride. The crude pentafluoroethane obtained after the crude purifying step was analyzed, whereupon it had the following composition:

| | | | |
|---|---|---|---|
| CF₃CHF₂ | 86.9712 | CF₃CHClF | 3.8204 |
| CF₃CHCl₂ | 0.0051 | CF₃CClF₂ | 0.3121 |
| CF₃CH₃ | 0.0161 | CH₂F₂ | 0.0121 |
| CF₂═CClF | 0.0241 | CF₂═CHF | 0.0012 |
| HF | 8.3276 | HCl | 0.4820 |
| Other | 0.0281 | | |
| | | | Unit: vol % |

### CATALYST PREPARATION EXAMPLE 1 (CATALYST EXAMPLE 1)

0.6 liter of pure water was poured into a 10-liter vessel and stirred. A solution obtained by dissolving 452 g of Cr(NO₃)₃.9H₂O and 42 g of In(NO₃)₃.nH₂O (n is about 5) in 1.2 liter of pure water and 0.31 liter of 28% aqueous ammonia were dropped into the vessel over about 1 hour while controlling the flow rate of the two types of aqueous solutions so that the pH of the reaction solution became a range from 7.5 to 8.5. The obtained slurry was filtered, then the filtered solids were washed well by pure water and dried at 120° C. over 12 hours. The dried solid was crushed, then mixed with graphite, and processed by a tablet-making machine to prepare pellets. This pellets were calcined at 400° C. for 4 hours under a flow of nitrogen gas to obtain a catalyst precursor. Next, the catalyst precursor was filled in an Inconel reactor where hydrogen fluoride was used for fluorination (activation of catalyst) at 350° C. to thereby prepare the catalyst.

### CATALYST PREPARATION EXAMPLE 2 (CATALYST EXAMPLE 2)

191.5 g of chromium chloride (CrCl₃.6H₂O) was placed in 132 ml of pure water which was then heated to 70 to 80° C. on a bath to dissolve the chromium chloride. The solution was cooled to room temperature, then 400 g of active alumina (NST-7 made by Nikki Universal Co. Ltd.) was dipped in it to make the alumina absorb the total amount of the catalytic solution. Then, the alumina wet by the catalytic solution was dried on a 90° C. bath and dried to a solid. The solidified catalyst was dried at 110° C. for 3 hours by an air circulation type hot air drier, the dry catalyst was filled in a vessel made by SUS, then the temperature was raised to 400° C. under the circulation of air to prepare the catalyst precursor. The fluorination of the catalyst (activation of the catalyst) was performed by the same procedure and under the same conditions as in Catalyst Preparation Example 1 to prepare the catalyst.

### CATALYST PREPARATION EXAMPLE 3 (CATALYST EXAMPLE 3)

The same procedure and operation as in Catalyst Preparation Example 2 were performed to prepare a catalyst except for adding 16.6 g of zinc chloride (ZnCl₂) into Catalyst Example 2 as the second ingredient.

### COMPARATIVE EXAMPLE

80 ml of the catalyst obtained in Catalyst Preparation Example 1 (Catalyst Example 1) was filled in an Inconel 600 type reactor having an inside diameter of 1 inch and a length of 1 m. The temperature in the reactor was held at 180° C. in a flow of nitrogen gas, crude 1,1,1,2-tetrafluoroethane (Material Example 1) was introduced into the reactor, then the feed of nitrogen gas was stopped. Only the crude 1,1,1,2-tetrafluoroethane was fed into the catalyst at 72 NL/hr. After the elapse of about 4 hours, the exhaust gas was stripped of the acid component by an aqueous alkali solution, then the gas composition was analyzed using a gas chromatograph. The gas had the following composition:

| | | | |
|---|---|---|---|
| CF₃CH₂F | 90.2993 | CHCl═CHF | 0.0003 |
| CF₃CH₂Cl | 7.6247 | CF₃CH₃ | 0.6260 |
| CF₃CHF₂ | 0.5916 | CF₃CHClF | 0.5904 |
| CHF₂CHF₂ | 0.1779 | CF₃CClF₂ | 0.0601 |
| CF₂═CHCl | 0.0278 | CH₂ClCHF₂ | 0.0019 |
| | | | Unit: vol % |

As apparent from the above analysis results, the conversion rate of the unsaturated compounds in the 1,1,1,2-tetrafluoroethane was about 95.8%, so it was proved that they could not be completely eliminated.

Next, the reaction was continued under the above conditions. After the elapse of 2400 hours, the composition of the exhaust gas was analyzed. As a result, it was confirmed that the content of CF₂═CHCl increased. The conversion rate of the unsaturated compounds was lowered to about 93%. The reaction was stopped at this point of time, the catalyst was extracted, and the surface was observed, whereupon deposition of carbon (black) on the catalyst surface was confirmed.

### EXAMPLE 1

An Inconel 600 type reactor having an inside diameter of 1 inch and a length of 1 m was filled with 80 ml of the catalyst obtained in Catalyst Preparation Example 1 (Catalyst Example 1) in the same way as the Comparative Example, the temperature in the reactor was held at 180° C. while passing nitrogen gas, hydrogen fluoride was fed from the inlet of the reactor at 10 NL/hr, then crude 1,1,1,2-tetrafluoroethane (Material Example 1) was fed into the reactor at 72 NL/hr, then the feed of the nitrogen gas was stopped. After the elapse of 4 hours, the exhaust gas was stripped of the acid component by an aqueous alkali solution, then the gas composition was analyzed by a gas chromatograph. It had the following composition:

| | | | |
|---|---|---|---|
| CF₃CH₂F | 90.2998 | CHCl═CHF | <0.0001 |
| CF₃CH₂Cl | 7.6524 | CF₃CH₃ | 0.6259 |
| CF₃CHF₂ | 0.5918 | CF₃CHClF | 0.5902 |
| CHF₂CHF₂ | 0.1777 | CF₃CClF₂ | 0.0600 |
| CF₂═CHCl | <0.0001 | CH₂ClCHF₂ | 0.0020 |
| | | | Unit: vol % |

As apparent from the analysis results, the conversion rate of the unsaturated compounds became about 99.9% by newly adding hydrogen fluoride to the crude 1,1,1,2-tetrafluoroethane.

Next, the gas after being stripped of the acid component by the above aqueous alkali solution was collected while cooling a cylinder and distilled to cut the low boiling point fraction and cut the high boiling point fraction to obtain high purity 1,1,1,2-tetrafluoroethane. The purity was analyzed by gas chromatography (TCD method or FID method) and a gas chromatography-mass spectrometer (GC-MS method). It had the following composition:

| | | | |
|---|---|---|---|
| CF₃CH₂F | 99.9956 | CHF₂CHF₂ | 0.0042 |

| | |
|---|---|
| Chlorine-containing compounds | <0.0002 |
| | Unit: vol % |

As apparent from the results, the chlorine-containing compounds were contained in the 1,1,1,2-tetrafluoroethane in an amount of 2 volppm or less. If combined with the isomer 1,1,2,2-tetrafluoroethane, the purity became about 99.999 vol % or more.

Further, when continuing the purifying reaction of the crude 1,1,1,2-tetrafluoroethane under the same conditions and analyzing the composition of the exhaust gas after the elapse of 2400 hours, no increase of CF₂═CHCl as seen in the Comparative Example was confirmed. The conversion rate of the unsaturated compound was also maintained at about 99% or more.

When the reaction was stopped at this point of time in the same way as Comparative Example and the catalyst extracted and its surface observed, no deposition of carbon was confirmed. Thereafter, the catalyst was filled again in a reactor and the reaction continued for 2000 hours under the same conditions, but the conversion rate of the unsaturated compounds was maintained at about 99% or more.

### EXAMPLE 2

Nitrogen gas was supplied through an Inconel 600 reactor (electric heater heating type: passivation by fluorine gas at temperature of 500° C. finished) having an inside diameter of 20.6 mm and a length of 500 mm at 30 NL/hr, and the temperature was elevated to 280° C. Then, as the diluting gas, the hydrogen fluoride was supplied at 50 NL/hr. Further, the 1,1,1,2-tetrafluoroethane obtained in Example 1 was supplied to one of the gas streams of the branched diluting gas at 1.8 NL/hr. Thereafter, fluorine gas was supplied to another gas stream of the diluting gas branched in the same way as the above at 2.7 NL/hr, and the reaction was carried out. After the elapse of 3 hours, the reaction gas was stripped of the hydrogen fluoride and the fluorine gas by an aqueous potassium hydroxide solution and an aqueous potassium iodide solution, then was analyzed for composition by a gas chromatograph. The gas composition was as follows:

| | | | |
|---|---|---|---|
| CF₄ | 0.4870 | CF₃CF₃ | 49.6001 |
| CF₃CHF₂ | 49.9126 | CF₃CH₂F | <0.0001 |

| | |
|---|---|
| Chlorine-containing compounds | <0.0002 |
| | Unit: vol % |

Next, the gas after being stripped of the hydrogen fluoride and fluorine gas was collected while cooling the cylinder and distilled to separate the CF₃CF₃ and CF₃CHF₂. Their low boiling point fractions and high boiling point fractions were cut, then the results were analyzed by a gas chromatograph and GC-MS. The purity of the CF₃CF₃ was 99.9999 vol % or more, and the purity of the CF₃CHF₂ was 99.9998 vol %, so high purity products could be acquired.

### EXAMPLE 3

An Inconel 600 type reactor having an inside diameter of 1 inch and a length of 1 m was filled with 80 ml of the catalyst obtained in Catalyst Preparation Example 2 (Catalyst Example 2). The temperature in the reactor was held at 180° C. while supplying nitrogen gas, the hydrogen fluoride was fed from the inlet of the reactor at 10 NL/hr, the crude pentafluoroethane (Material Example 2) was fed into the reactor at 72 NL/hr, then the feed of the nitrogen gas was stopped. After the elapse of 4 hours, the exhaust gas was stripped of its acid component by an aqueous alkali solution and analyzed by using a gas chromatograph. It had the following composition:

| | | | |
|---|---|---|---|
| CF₃CHF₂ | 95.3734 | CF₃CHClF | 4.2156 |
| CF₃CHCl₂ | 0.0056 | CF₃CClF₂ | 0.3422 |
| CF₃CH₃ | 0.0176 | CH₂F₂ | 0.0133 |
| CF₂═CClF | <0.0002 | CF₂═CHF | <0.0001 |
| CF₃CH₂F | 0.0012 | Other | 0.0308 |
| | | | Unit: vol % |

As apparent from the above results, about 99% of the unsaturated compounds in the crude pentafluoroethane could be removed (converted).

### EXAMPLE 4

An Inconel 600 type reactor having an inside diameter of 1 inch and a length of 1 m was filled with 80 ml of the catalyst obtained in Catalyst Preparation Example 3 (Catalyst Example 3). The temperature in the reactor was held at 180° C. while supplying nitrogen gas, hydrogen fluoride was fed from the inlet of the reactor at 10 NL/hr, then 36 NL/hr of the crude 1,1,1,2-tetrafluoroethane (Material Example 1) and the 36 NL/hr of the crude pentafluoroethane (Material Example 2) were mixed at the inlet of the reactor and fed into the reactor. Thereafter, the feed of the nitrogen gas was stopped. After the elapse of 4 hours, the exhaust gas was stripped of its acid component by an aqueous alkali solution, the gas composition was analyzed by a gas chromatograph. About 99% of the contained unsaturated compounds could be removed (converted).

### INDUSTRIAL APPLICABILITY

The present invention is useful for the production of 1,1,1,2-tetrafluoroethane and pentafluoroethane which can be advantageously utilized as a low temperature use refrigerant, an etching gas, and a cleaning gas.

1. A process for producing high purity 1,1,1,2-tetrafluoroethane and/or pentafluoroethane by a step of purifying a crude product obtained by reacting trichloroethylene and/or tetrachloroethylene with hydrogen fluoride comprised of a main product including 1,1,1,2-tetrafluoroethane and/or pentafluoroethane, hydrogen fluoride as an azeotropic component with the main product, and impurity ingredients including at least an unsaturated compound, wherein said purifying step includes a step of bringing a mixture obtained by newly adding hydrogen fluoride into said crude product into contact with a fluorination catalyst in the vapor phase to reducing the content of the unsaturated compound contained in said crude product and a distillation step.

2. A production process as set forth in claim 1, wherein the content of the hydrogen chloride contained as the impurity in said crude product is 2 mol % or less.

3. A production process as set forth in claim 1, wherein the concentration of the 1,1,1,2-tetrafluoroethane and/or pentafluoroethane contained in said crude product is 70 mol % or more.

4. A production process as set forth in claim 1, wherein said unsaturated compound is at least one compound selected from a group consisting of 1,1-difluoro-2-chloroethylene, 1,2-difluoro-1-chloroethylene, 1-chloro-2-fluoroethylene, 1,1,2-trifluoroethylene, and 1-chloro-1,2,2-trifluoroethylene.

5. A production process as set forth in claim 1, wherein said fluorination catalyst includes at least one metal element selected from a group consisting of Cu, Mg, Zn, Pb, V, Bi, Cr, In, Mn, Fe, Co, Ni, and Al.

6. A production process as set forth in claim 1, wherein a contact temperature between said mixture and said fluorination catalyst is within a range of from 130 to 280° C.

7. A production process as set forth in claim 1, wherein a mixture obtained by newly adding hydrogen fluoride to a crude product comprised of a main product including 1,1,1,2-tetrafluoroethane, hydrogen fluoride as an azeotropic component with the main product, and impurity ingredients including at least an unsaturated compound is brought into contact with the fluorination catalyst in the vapor phase to reduce the content of the unsaturated compound contained in said crude product.

8. A production process as set forth in claim 7, wherein the contact temperature between said mixture and said fluorination catalyst is within a range of from 130 to 200° C.

9. A production process as set forth in claim 1, further comprising separating the hydrogen fluoride in said distillation step and recirculating the separated hydrogen fluoride to a step for obtaining said crude product.

10. A 1,1,1,2-tetrafluoroethane obtained by the production process as set forth in claim 1, wherein a total content of chlorine-containing compounds in said 1,1,1,2-tetrafluoroethane is 2 volppm or less.

11. A process for production of pentafluoroethane and/or hexafluoroethane comprising reacting the 1,1,1,2-tetrafluoroethane as set forth in claim 10 and fluorine gas in the presence of a diluting gas.

12. An etching gas comprising pentafluoroethane and/or hexafluoroethane obtained by the production process as set forth in claim 11.

13. A cleaning gas comprising pentafluoroethane and/or hexafluoroethane obtained by the production process as set forth in claim 11.
